(19) United States
(12) Patent Application Publication
(10) Pub. No.: US 2023/0277826 A1
(43) Pub. Date: Sep. 7, 2023

(54) INACTIVATED VIRUS VACCINE MICRONEEDLE PRODUCT AS WELL AS PREPARATION METHOD AND APPLICATION THEREOF

(71) Applicants: Healthina Stem Cell Industry Platform (Tianjin) Limited, Tianjin (CN); Tangyi Holdings(Shenzhen) Limited, Shenzhen (CN)

(72) Inventors: Bin ZHENG, Tianjin (CN); Yulin CAO, Beijing (CN); Wei SUN, Tianjin (CN); Shixiang CHENG, Tianjin (CN)

(73) Assignees: Healthina Stem Cell Industry Platform (Tianjin) Limited, Tianjin (CN); Tangyi Holdings(Shenzhen) Limited, Shenzhen (CN)

(21) Appl. No.: 17/750,309

(22) Filed: May 21, 2022

(30) Foreign Application Priority Data
Mar. 2, 2022 (CN) 202210198246.9

(51) Int. Cl. A61M 37/00; A61K 9/00; C12N 7/00

(52) U.S. Cl. CPC A61M 37/0015; A61K 9/0021; C12N 7/00; A61M 2202/30; A61M 2037/0053; C12N 2796/00

(57) ABSTRACT

An inactivated virus vaccine microneedle product includes a backing and an inactivated virus-containing microneedle array attached to a side of the backing, wherein the inactivated virus-containing microneedle array includes a plurality of microneedles, and each microneedle contains a matrix and an inactivated virus loaded in the matrix. The present invention adopts the inactivated virus vaccine microneedle product as well as a preparation method and an application thereof, and such microneedle product realizes efficient transdermal absorption of a vaccine by loading an inactivated virus in the microneedle product after being administered to the skin, and a long-acting stable release of a vaccine is achieved, which solves the problems of traditional vaccination, such as muscular pain and multiple injections.

### CROSS REFERENCE TO THE RELATED APPLICATIONS

This application is based upon and claims priority to Chinese Patent Application No. 202210198246.9, filed on Mar. 2, 2022, the entire contents of which are incorporated herein by reference.

### TECHNICAL FIELD

The present invention relates to the technical field of biological microneedles, in particular to an inactivated virus vaccine microneedle product as well as a preparation method and an application thereof.

### BACKGROUND

The virus epidemic around the world is still very serious, and has resulted in immeasurable losses to the economy and people's lives of all countries. As the virus epidemic is gradually controlled, widespread vaccination plays a relatively important role. Current virus pneumonia vaccines include inactivated vaccines, recombinant subunit vaccines, adenovirus vector vaccines and nucleic acid vaccines, which are mainly administrated by intramuscular injection.

However, the intramuscular injection presents some insurmountable problems. For example, cold chain storage is required, and cold chain storage greatly increases the costs of transportation, storage and use; the intramuscular injection requires a certain injection dose to take effect, generally one or two or more injections are administrated, and both the use of medical resources and the time arrangement of vaccinees are problematic.

Served as a new technique of locally transdermal administration, microneedle administration combines convenience of emplastrum and effectiveness of subcutaneous injection, avoids the shortcomings of other administration methods, and features the advantages of no nerve access, safety, no pain, efficient penetration, etc. However, it is difficult to store a virus vaccine in microneedle patches in the prior art.

### SUMMARY

The present invention is intended to provide an inactivated virus vaccine microneedle product as well as a preparation method and an application thereof, wherein such microneedle product realizes efficient transdermal absorption of a vaccine by loading an inactivated virus in the microneedle product after being administered to the skin, and a long-acting stable release of a vaccine is achieved.

To achieve the aforesaid purposes, the present invention provides an inactivated virus vaccine microneedle product, including a backing and an inactivated virus-containing microneedle array attached to a side of the backing, wherein the inactivated virus-containing microneedle array includes a plurality of microneedles, wherein each microneedle contains a matrix and an inactivated virus loaded in the matrix.

Preferably, each of the microneedles includes a tip and a bottom, wherein the tip is away from the backing, each microneedle is attached to the backing via the bottom, a distance from the tip to the bottom is 200 μm-1 mm, a diameter of the bottom is 100 μm-500 μm, and a spacing between the adjacent tips is 300 μm-800 μm.

Preferably, an inactivated virus is cultured on a cell matrix, and then infectious viruses therein are killed by a physical or chemical method while an integrity of antigen particles of such virus is maintained, so that the virus loses pathogenicity and retains antigenicity.

Preferably, the matrixes are formed by crosslinking and/or drying and curing of an aqueous solution containing one or more of the following substances: polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, polyvinylpyrrolidone (PVP) or polyvinyl alcohol, preferably GelMA and/or hyaluronic acid, and more preferably GelMA.

Preferably, the backing is formed by crosslinking and/or drying and curing of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP and polyvinyl alcohol, and preferably hyaluronic acid.

A method for preparing the inactivated virus vaccine microneedle product, including the following steps:

(1) preparing an inactivated virus: culturing an inactivated virus vaccine virus on a cell matrix, and then killing infectious viruses therein by a physical or chemical method while maintaining an integrity of antigen particles of such virus, so that the virus loses pathogenicity and retains antigenicity;

(2) adding a material forming a matrix to the inactivated virus-containing liquid medium obtained in step (1) to form a mixed solution; wherein in the mixed solution, the matrix material accounts for 20%-40% of the mixed solution based on weight percentages, preferably 25%-35%, and more preferably 30%;

(3) placing the mixed solution obtained in step (2) in molding holes of a microneedle mold, and filling at least a portion of the volume of the molding holes, and preferably the whole volume of the molding holes;

(4) crosslinking the mixed solution containing the matrix material and the inactivated virus in the molding holes to form microneedles in the molding holes, wherein a plurality of microneedles form an inactivated virus vaccine microneedle array;

(5) applying a backing material-containing solution to bottom surfaces of the microneedles and an upper surface of the microneedle mold that is not covered by the microneedles to form a backing solution layer, and crosslinking the backing material to form a continuous backing layer, so that the inactivated virus vaccine microneedle array is attached to the backing solution layer or the backing layer; and

(6) simultaneously drying and curing the backing solution layer or the backing layer obtained in step (5) and the inactivated virus vaccine microneedle array to form an inactivated virus vaccine microneedle product.

Preferably, in step (3), the microneedle mold includes an upper surface and molding holes extending downward from the upper surface, wherein each of the molding holes preferably includes a tip and a bottom, the tip is away from the upper surface, the bottom plane is flush with the upper surface, a distance from the tip to the bottom is 200 μm-1 mm, a diameter of the bottom is 100 μm-500 μm, and a spacing between the adjacent tips is 300 μm-800 μm.

Preferably, in step (4), ultraviolet crosslinking is preferably performed for 5 s-15 s, and preferably 10 s, and/or drying and curing are performed, and hot curing is preferably performed.

The present invention further provides an application of the inactivated virus vaccine microneedle product in preparing medical devices or drugs against virus pneumonia.

Therefore, the present invention adopts the inactivated virus vaccine microneedle product as well as a preparation method and an application thereof. With full use of the advantages of the microneedle product and inactivated vaccine, the microneedle product can realize rapid humoral immunity by loading an inactivated virus vaccine in the microneedle product after being administered to the skin, and antibodies produced by such microneedle product have the effects of neutralizing and removing pathogenic microorganisms and toxins therein, and have an excellent protective effect on the pathogenic microorganisms of extracellular infection. Moreover, a virus vaccine present in the microneedle patch can not only work for a long time, but also reduce the discomfort caused by intramuscular injection and the use of medical resources.

A microneedle product generally includes a plurality of microneedles with a length of no more than 1 mm. The microneedles can form micro channels in the skin cuticle, break through the barrier of the skin cuticle and promote the penetration of drugs, thus reducing a dose of drugs accumulated in the cuticle and increasing a dose of drugs reaching the epidermal, dermal and subcutaneous tissues.

The present invention features the following specific technical effects:

(1) Antibodies produced by the inactivated virus vaccine microneedle product of the present invention have the effects of neutralizing and removing pathogenic microorganisms and toxins therein, and have an excellent protective effect on the pathogenic microorganisms of extracellular infection.

(2) The inactivated virus vaccine microneedle product of the present invention can pierce the skin cuticle which limits drug absorption, and promote the spread of inactivated virus without causing pain.

(3) Microneedles are prepared by a microneedle template reverse mold. The method is simple, easy to operate, low-cost, reusable, easy to control the basic shape of a microneedle array, highly safe, and suitable for promotion, without needing high technical requirements.

The technical solutions of the present invention will be further described below in detail in combination with the accompanying drawings and embodiments.

### BRIEF DESCRIPTION OF THE DRAWINGS

FIG. 1 is a structural side view of a part of an inactivated virus vaccine microneedle product.

FIG. 2 is a structural side view of a part of a microneedle mold of an inactivated virus vaccine microneedle product.

### LABELS IN THE FIGURES

100. inactivated virus vaccine microneedle product; 110. microneedle; 120. backing; 200. microneedle mold; 201. molding hole; 202. upper surface.

### DETAILED DESCRIPTION OF THE EMBODIMENTS

The technical solutions of the present invention will be further described below in combination with the accompanying drawings and embodiments.

Unless otherwise defined, the technical or scientific terms used herein should have ordinary meanings understood by those of ordinary skill in the art of the present invention.

It is apparent to those skilled in the art that the present invention is not limited to the details of the above-mentioned exemplary embodiments and can be realized in other specific forms without departing from the intention or essential features of the present invention. Therefore, in all respects, the embodiments should be considered to be exemplary and non-restrictive. The scope of the present invention is limited by the appended claims rather than the above-mentioned description, so that all changes falling within the meaning and scope of the equivalents of the claims are intended to be included in the present invention, and any accompanying drawing marks in the claims should not be deemed to limit the claims involved.

Moreover, it should be understood that although the specification is described according to the implementation modes, not each implementation mode contains only one independent technical solution. This narrative form of the specification is for the sake of clarity only. Those skilled in the art should take the specification as a whole, and the technical solutions in various embodiments may be combined appropriately to form other implementation modes that can be understood by those skilled in the art. These other implementation modes should also fall within the protection scope of the present invention.

In addition, it should be understood that the above-mentioned specific embodiments are used for explaining the present invention only, and the protection scope of the present invention is not limited to such specific embodiments. Within the technical scope disclosed by the present invention, the equivalent substitutions or changes made by those skilled in the art based on the technical solutions and inventive concept of the present invention should fall within the protection scope of the present invention/invention.

The “including/comprising” or “containing” and similar words used herein refer to that the element ahead of the word covers the elements listed behind the word and does not exclude the possibility of covering other elements as well. The orientations or position relations indicated by terms “inside”, “outside”, “up” and “down” are those shown based on the accompanying drawings, only used for the convenience of describing the present invention and simplifying the description, rather than indicating or implying that the device or element referred to must have a particular orientation or be constructed and operated in a particular orientation, so they cannot be understood as a limitation to the present invention. When the absolute position of the object described changes, the relative position relation may also change accordingly. In the present invention, unless otherwise expressly specified and limited, the term “attaching” should be understood in a broad sense. For example, two elements may be connected fixedly, connected detachably, or integrated; two elements may be connected directly, or connected indirectly through an intermediate medium, or communicated internally or interact. Those of ordinary skill in the art can understand the specific meanings of such terms in the present invention according to the specific situations. The term “about” used herein has the meaning known to those skilled in the art, and preferably refers to that the value modified by the term is within the range of ±50%, ±40%, ±30%, ±20%, ±10%, ±5% or ±1%.

All terms (including technical or scientific terms) used in the disclosure have the same meanings as those understood by those of ordinary skill in the art of the disclosure, unless otherwise specifically defined. Moreover, it should be understood that terms defined in a general dictionary should be understood to have meanings consistent with those in the context of the relevant techniques, and should not be interpreted in an idealized or highly formal sense, unless expressly defined herein.

The techniques, methods and equipment known to those of ordinary skill in the art may not be discussed in detail, but where appropriate, such techniques, methods and equipment should be considered as a part of the specification.

The contents disclosed in the prior art literature referenced in the specification of the present invention are incorporated herein by reference in its entirety.

### Example 1

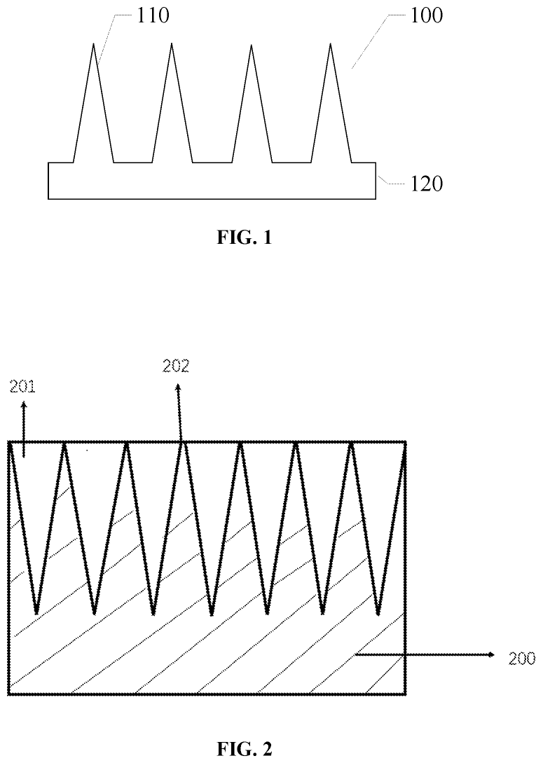

As shown in FIG. 1, the inactivated virus vaccine microneedle product 100 of the present invention includes a backing 120 and an inactivated virus vaccine microneedle array attached to a side of the backing 120, wherein the inactivated virus vaccine microneedle array includes a plurality of microneedles 110, wherein each microneedle 110 contains a matrix and an inactivated virus loaded in the matrix.

There is no special limitation to a matrix material forming a matrix, and all common matrix materials for preparing the microneedle product adopted in the field can be applied in the present invention. However, considering that the formed microneedles 110 need to have a certain mechanical strength, and the cured microneedles 110 need to have a certain porosity, the matrixes are formed by crosslinking and/or drying and curing of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP and polyvinyl alcohol, preferably GelMA and/or hyaluronic acid, and more preferably GelMA.

Herein, the matrix material is preferably GelMA and/or hyaluronic acid. The GelMA is prepared from methacrylic anhydride (MA) and gelatin, which are crosslinked by UV light under the mediation of photosensitizer to form a pore structure with a certain strength. The GelMA features excellent biocompatibility. The hyaluronic acid is a mucopolysaccharide, which has a function of skin protection and can be applied to accelerate wound healing.

There is no special limitation to a backing material forming a backing 120, and all common backing materials for preparing the microneedle product adopted in the field can be applied in the present invention. However, considering that the formed backing 120 needs to have a certain mechanical strength and flexibility, the backing 120 is preferably formed by crosslinking and/or drying of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP and polyvinyl alcohol, and preferably hyaluronic acid.

The hyaluronic acid is a mucopolysaccharide, which has a function of skin protection and can be applied to accelerate wound healing, so the backing material is preferably hyaluronic acid.

There is no limitation to a thickness of the formed backing 120, but considering that the backing 120 needs to have a certain strength and flexibility, such thickness is preferably 0.1-15 mm, more preferably 1-10 mm, and most preferably 2-3 mm.

Further, in some cases, in the inactivated virus vaccine microneedle product 100, materials forming the matrixes and the backing 120 are the same. In these cases, the microneedles 110 and the backing 120 are combined more stably, and in the following method for preparing the inactivated virus vaccine microneedle product 100, the microneedle array and the backing 120 can be integrated, simplifying a preparation process. In other cases, the materials of the matrixes and the backing 120 are different; e.g., the matrix material is GelMA, while the backing material is hyaluronic acid. In such cases, the microneedles 110 have a certain strength, a pore structure and excellent biocompatibility; in addition, the backing 120 has a better protective effect on the skin, and can be applied to accelerate wound healing.

It should be noted that those skilled in the art can appropriately select matrix and backing materials according to application situations.

Further, in the inactivated virus vaccine microneedle product 100, there is no special limitation to the sizes and shapes of the microneedles 110, and both can vary over a wide range according to an application part of the inactivated virus vaccine microneedle product 100 and a disease to be treated.

As shown in FIG. 1, in the inactivated virus vaccine microneedle product 100, each microneedle 110 includes a tip and a bottom, wherein the tip is away from the backing 120, each microneedle 110 is attached to the backing 120 via the bottom, and there is no special limitation to a height between the tip and the bottom, but preferably 200 μm-1 mm. The height is preferably no lower than 200 μm, otherwise the microneedles 110 will not pierce some animal bodies, preferably the skin cuticles of some parts of a human body. However, the height is preferably no higher than 1 mm, otherwise the microneedles will pierce some animal bodies, preferably the skin cuticles of some parts of a human body, and reach the nervous layer, thus causing pain.

Further, the bottom of each of the microneedles 110 has a diameter of 100 μm-500 μm. The diameter of the bottom is preferably no less than 100 μm, otherwise the microneedles 110 will be easy to break due to insufficient mechanical strength. In addition, the diameter of the bottom is preferably no more than 500 μm, otherwise after the inactivated virus vaccine microneedle product 100 is applied to some animal bodies, preferably some parts of a human body, larger holes will be left on the skin, leading to problems concerning skin beauty and healing.

There is no special limitation to a stereo shape of the microneedles 110, and the microneedles can be in a shape of cylinder, cone, circular truncated cone, etc., or a combination of them, and preferably regular or irregular circular cone, conoid, triangular pyramid, rectangular pyramid or higher-level pyramid which may be a right cone or an oblique cone.

Further, a spacing between the adjacent tips of the microneedles 110 is 300 μm-800 μm. The spacing within such range can make the inactivated virus vaccine microneedle product 100 achieve an optimization effect in a depth of piercing the cuticle, etc.

It should be noted that, those skilled in the art can appropriately select shapes, sizes, etc. of the microneedles 110 in the inactivated virus vaccine microneedle product 100 according to practical application situations.

### Example 2

A method for preparing the inactivated virus vaccine microneedle product includes the following steps:

(1) An inactivated virus vaccine virus was prepared: An inactivated virus vaccine virus was cultured on a cell matrix, and then infectious viruses therein were killed by a physical or chemical method while an integrity of antigen particles of such virus was maintained, so that the virus lost pathogenicity and retained antigenicity.

(2) A matrix material forming a matrix was added to the inactivated virus vaccine virus-containing solution obtained in step (1) to form a mixed solution.

In step (2), there is no special limitation to a concentration of the matrix material in the mixed solution, as long as the microneedles 110 can be formed. Wherein the matrix material accounts for 20%-40% of the liquid medium based on weight percentages, preferably 25%-35%, and more preferably 30%. In such ranges, the microneedles 110 formed by the matrix material have sufficient mechanical strength and a certain porosity to achieve an optimum hydrogen generation efficiency.

(3) A microneedle mold 200 was provided. As shown in FIG. 2, the microneedle mold 200 includes an upper surface 202 and molding holes 201 extending downward from the upper surface 202, wherein each of the molding holes 201 includes a tip and a bottom, the tip is away from the upper surface 202, and the bottom plane is flush with the upper surface 202.

In step (3), a stereo shape of the molding holes 201 in the microneedle mold 200 should be matched with the expected shape of the microneedles 110. As above, the molding holes can be in a shape of cylinder, cone, circular truncated cone, etc., or a combination of them, and preferably regular or irregular circular cone, conoid, triangular pyramid, rectangular pyramid or higher-level pyramid which may be a right cone or an oblique cone.

Each of the molding holes 201 should have a height, a bottom width and a tip spacing corresponding to those of the microneedles 110. However, in some cases, each of the molding holes 201 can further have a height and a bottom width larger than those of the microneedles 110. In the latter case, the formed microneedles 110 will not fill the whole space of the molding holes 201.

Further, the upper surface 202 (including inner surface of the molding holes 201) of the microneedle mold 200 was coated with an anti-bonding layer.

In the present invention, the microneedle mold 200 is commercially available; for example, it can be a customized PDMS mold purchased from Taizhou Chipscreen Medical Technology Company, and the mold parameters can be customized according to the needs of the needle body size. Specifically, in the microneedle mold 200 adopted in the example of the present invention, the heights of all molding holes 201 were 600 μm, the bottom widths were 320 μm, the tip spacings were 500 μm, and the overall microneedle mold 200 was L×W=15 mm×15 mm.

(4) The mixed solution obtained in step (2) was placed in the molding holes 201 and filled at least a portion of the volume of the molding holes 201.

In step (4), there is no special limitation to the volume of the molding holes 201 filled by the mixed solution, but preferably at least ¼, ⅓, ½, ⅔ and ¾ of the volume of the molding holes 201 is filled by the mixed solution, and most preferably the whole volume is filled by the mixed solution. When the whole volume of the molding holes 201 is not filled, the portion of the molding holes 201 that is not filled by the microneedles 110 should be filled by a backing material solution in the step (6) below.

(5) The mixed solution containing the matrix material and the inactivated virus in the molding holes 201 was crosslinked and/or dried and cured to form microneedles 110 in the molding holes 201, and a plurality of microneedles 110 formed an inactivated virus vaccine microneedle array.

(6) A backing material-containing solution was applied to bottom surfaces of the microneedles 110 and the upper surface 202 of the microneedle mold 200 that was not covered by the microneedles 110 to form a backing solution layer, so that the inactivated virus vaccine microneedle array was attached to the backing solution layer.

(7) The backing solution layer obtained in step (6) and optional microneedle array simultaneously attached to the backing solution layer were dried and cured to form an inactivated virus vaccine microneedle product 100.

In step (6) and step (7), a water content in the microneedles 110 and the backing 120 in the drying process is preferably lower than 20%, more preferably lower than 10%, and most preferably lower than 5%.

The technical features of the inactivated virus vaccine microneedle product are as shown in example 1.

### Example 3

An application of the inactivated virus vaccine microneedle product prepared in example 1 and the method for preparing the inactivated virus vaccine microneedle product in example 2 in preparing medical devices and drugs against pneumonia.

In such example, the technical features and optimal ranges of the inactivated virus vaccine microneedle product and the preparation method thereof are still applicable.

### Example 4

The inactivated virus vaccine microneedle product is prepared according to a general process, including the following steps:

(1) an inactivated virus vaccine was prepared: an inactivated virus vaccine virus was cultured on a cell matrix, and then infectious viruses therein were killed by a physical or chemical method while an integrity of antigen particles of such virus was maintained, so that the virus lost pathogenicity and retained antigenicity;

(2) a matrix material (GelMA or HA) was added to the inactivated virus vaccine-containing solution obtained in step (1) to form a mixed solution, and 1% 2,4-dihydroxy benzophenone was added;

(3) a microneedle mold was provided;

(4) the mixed solution obtained in step (2) was placed in molding holes of the microneedle mold and filled the whole space of the molding holes;

(5) the mixed solution containing the matrix material and the inactivated virus vaccine solution in the molding holes was crosslinked, and ultraviolet crosslinked for 10 s, to form microneedles in the molding holes, and a plurality of microneedles formed an inactivated virus vaccine microneedle array. Wherein each microneedle includes a tip and a bottom, and the tip is away from the upper surface relative to the bottom;

(6) a backing material-containing solution was applied to bottom surfaces of the microneedles and the upper surface of the microneedle mold that was not covered by the microneedles to form a backing solution layer, and the backing material was subjected to hot curing to form continuous backing, so that the inactivated virus vaccine microneedle array was attached to a side of the backing; and

(7) the backing and the microneedle array attached to a side of the backing obtained in step (6) were subjected to hot drying so that a water content was lower than 5%, to form an inactivated virus vaccine microneedle product.

Test

Seventy healthy mice with an approximate body weight of 40 g were randomly divided into 10 groups with 7 mice in each group, and subjected to infection test through a control group and a test group, wherein five groups of mice in the test group were respectively applied with the microneedle vaccines prepared in example 4, and five groups of mice in the control group were respectively applied with blank microneedle products, which were inactivated virus preparations containing no microneedle vaccine of the present invention.

The chest hair of the mice was scraped, and the microneedle vaccines and blank microneedle products were respectively attached to the chest of the mice. After 7 d of vaccine attachment, the microneedle patches were removed and the mice were infected with a mimicking virus. The EC50 of monoclonal antibody was tested by ELISA to determine the infection of the mice.

The microneedle patches prepared by each group were applied to the skins of the points corresponding to the lungs of mice, and the mice were infected with the virus. After 2 d of normal feeding, the survival was observed, as shown in Table 1.

TABLE 1
Survival of Mice Infected with Viruses of Microneedles of Control Group and Test Group

| | Group | | | | | | | | | |
|---|---|---|---|---|---|---|---|---|---|---|
| | Control Group | | | | | Test Group | | | | |
| Effect | 1 | 2 | 3 | 4 | 5 | 1 | 2 | 3 | 4 | 5 |
| IgG Log10 (ELISA EC50) | 0 | 0 | 0.6 | 0 | 0 | 1.4 | 1.6 | 1.4 | 2.3 | 1.5 |
| Body weight (g) of mice | 0 | 0 | 40 | 0 | 0 | 45 | 44 | 41 | 44 | 47 |
| Clinical score of mice | 0 | 0 | 6 | 0 | 0 | 7 | 7 | 6 | 7 | 7 |
| Survival result of mice | Dead | Dead | Alive | Dead | Dead | Alive | Alive | Alive | Alive | Alive |

| | | |
|---|---|---|
| Survival rate of mice | 20% | 100% |

Therefore, the present invention adopts the inactivated virus vaccine microneedle product as well as the preparation method and the application thereof, wherein such microneedle product realizes efficient transdermal absorption of a vaccine by loading an inactivated virus in the microneedle product after being administered to the skin, and a long-acting stable release of a vaccine is achieved.

Finally, it should be stated that the above-mentioned embodiments are only used for describing, rather than limiting, the technical solutions of the present invention. Although the present invention is described in detail by reference to the preferred embodiments, those of ordinary skill in the art should understand that they can still make modifications or equivalent substitutions to the technical solutions of the present invention, but these modifications or equivalent substitutions will not make the modified technical solutions deviate from the spirit and scope of the technical solutions of the present invention.

1. An inactivated virus vaccine microneedle product, comprising a backing and an inactivated virus-containing microneedle array attached to a side of the backing, wherein the inactivated virus-containing microneedle array comprises a plurality of microneedles, wherein each of the plurality of microneedles comprises a matrix and an inactivated virus loaded in the matrix.

2. The inactivated virus vaccine microneedle product of claim 1, wherein each of the plurality of microneedles comprises a tip and a bottom, wherein the tip is away from the backing, each of the plurality of microneedles is attached to the backing via the bottom, a distance from the tip to the bottom is 200 μm-1 mm, a diameter of the bottom is 100 μm-500 μm, and a spacing between the adjacent tips is 300 μm-800 μm.

3. The inactivated virus vaccine microneedle product of claim 1, wherein the inactivated virus is cultured on a cell matrix, and then infectious viruses therein are killed by a physical or chemical method while an integrity of antigen particles of the inactivated virus is maintained, so that the inactivated virus loses pathogenicity and retains antigenicity.

4. The inactivated virus vaccine microneedle product of claim 1, wherein the matrix is formed by crosslinking and/or drying and curing of an aqueous solution containing at least one substance selected from the group consisting of: polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, polyvinylpyrrolidone (PVP), and polyvinyl alcohol, preferably GelMA and/or hyaluronic acid, and more preferably GelMA.

5. The inactivated virus vaccine microneedle product of claim 1, wherein the backing is formed by crosslinking and/or drying and curing of an aqueous solution containing at least one substance selected from the group consisting of: polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, polyvinylpyrrolidone (PVP), and polyvinyl alcohol, and preferably hyaluronic acid.

6. A preparation method of the inactivated virus vaccine microneedle product of claim 1, comprising the following steps:
1) preparing the inactivated virus: culturing the inactivated virus on a cell matrix, and then killing infectious viruses therein by a physical or chemical method while maintaining an integrity of antigen particles of the inactivated virus, so that the inactivated virus loses pathogenicity and retains antigenicity, and obtaining an inactivated virus-containing liquid medium;
2) adding a matrix material forming the matrix to the inactivated virus-containing liquid medium obtained in step 1 to form a mixed solution, wherein in the mixed solution, the matrix material accounts for 20%-40% of the mixed solution based on weight percentages, preferably 25%-35%, and more preferably 30%;
3) placing the mixed solution obtained in step 2 in molding holes of a microneedle mold, and filling at least a portion of a volume of the molding holes, and preferably a whole volume of the molding holes;
4) crosslinking the mixed solution containing the matrix material and the inactivated virus in the molding holes to form the plurality of microneedles in the molding holes, wherein the plurality of microneedles form an inactivated virus vaccine microneedle array;
5) applying a solution containing a backing material to bottom surfaces of the plurality of microneedles and an upper surface of the microneedle mold that is not covered by the plurality of microneedles to form a backing solution layer, and crosslinking the backing material in the backing solution layer to form a continuous backing layer, so that the inactivated virus vaccine microneedle array is attached to the backing solution layer or the continuous backing layer; and
6) simultaneously drying and curing the backing solution layer or the continuous backing layer obtained in step 5 and the inactivated virus vaccine microneedle array to form the inactivated virus vaccine microneedle product.

7. The preparation method of claim 6, wherein in step 3, the microneedle mold comprises an upper surface and molding holes extending downward from the upper surface, wherein each of the molding holes preferably comprises a tip and a bottom, the tip is away from the upper surface, a bottom plane is flush with the upper surface, a distance from the tip to the bottom is 200 μm-1 mm, a diameter of the bottom is 100 μm-500 μm, and a spacing between the adjacent tips is 300 μm-800 μm.

8. The preparation method of claim 6, wherein in step 4, ultraviolet crosslinking is performed for 5 s-15 s, and preferably 10 s, and/or the drying and curing are performed, and hot curing is performed.

9. An application of the inactivated virus vaccine microneedle product according to claim 1 in preparing medical devices or drugs against virus pneumonia.
